(19) United States
(12) Patent Application Publication
(10) Pub. No.: US 2012/0033870 A1
(43) Pub. Date: Feb. 9, 2012

(54) IMAGE DATA TRANSFER APPARATUS AND CONTROL METHOD FOR THE SAME

(75) Inventor: Hirokazu Ohguri, Funabashi-shi (JP)

(73) Assignee: CANON KABUSHIKI KAISHA, Tokyo (JP)

(21) Appl. No.: 13/191,915

(22) Filed: Jul. 27, 2011

(30) Foreign Application Priority Data
Aug. 9, 2010 (JP) 2010-179010

(51) Int. Cl. G06K 9/00

(52) U.S. Cl. 382/132

(57) ABSTRACT

An image data transfer apparatus for an X-ray imaging apparatus, transferring X-ray image data output from an X-ray detection unit to an external device, acquires pixel values that constitute the X-ray image data in a pixel order in which the X-ray detection unit outputs the X-ray image data. The image data transfer apparatus divides the X-ray image data into a predetermined number of reduced images by grouping each acquired pixel value according to a pixel position in an image, and holds each reduced image in a memory area with consecutive addresses. The image data transfer apparatus transfers the X-ray image data to the external device as each held reduced image.

### BACKGROUND OF THE INVENTION

1. Field of the Invention

The present invention relates to an image data transfer apparatus, and more particularly to an image data transfer apparatus capable of being suitably used for an X-ray imaging apparatus that irradiates an object with an X-ray to obtain X-ray image data according to the intensity of the X-ray transmitted through the object.

2. Description of the Related Art

Digital X-ray imaging apparatuses have been commercially available that irradiate an object with an X-ray from an X-ray irradiation source, digitize an X-ray image that is an intensity distribution of the X-ray transmitted through the object, and perform required image processing for the digitized X-ray image to generate a sharper X-ray image. Such a digital X-ray imaging apparatus transfers obtained X-ray image data to an image processing device such as a personal computer for the purposes of image processing and storage. The image processing device transfers the image-processed X-ray image data to an image viewing device such as a display to cause the X-ray image data to be displayed thereon. The transfer of the X-ray image data and other communications such as control signals between the X-ray imaging apparatus and the image processing device may be performed via a wired LAN or a wireless LAN.

If the image data transfer and other communications between the X-ray imaging apparatus and the image processing device are performed during the reading of the X-ray image data involved in the X-ray irradiation, noises caused by the communications may be introduced into the image data being read, thereby affecting the image quality. As such, a proposal has been made to avoid this inconvenience by transferring the X-ray image data from the X-ray imaging apparatus to the image processing device after the reading of the X-ray image data is finished, rather than transferring the X-ray image data in parallel with reading the X-ray image data (see Japanese Patent Laid-Open No. 2006-087566).

Unfortunately, the proposed approach requires some time for the X-ray image to be displayed on the display device after the X-ray image is taken, possibly impairing the usability for a user. As such, in another conventional approach, reduced image data generated from full-size image data (hereinafter referred to as full image data) is sent to the image processing device before the full image data is sent to the image processing device. The reduced image may be generated by thinning particular pixels from the full image (see Japanese Patent Laid-Open No. 2003-325494). Until the full image data is sent, the reduced image data subjected to image processing by the image processing device may be used to provide a preview display on the display device to reduce the waiting time for a user.

If the full image data is stored in its original form in memory, it means that pixel data required to generate the reduced image data does not reside at consecutive addresses in the memory. Therefore, to generate the reduced image data to be transferred, burst reading is performed and unnecessary pixels are discarded, or discrete addresses are accessed and only necessary pixels are read: this prevents efficient reading of the reduced image. The resulting redundant reading time may cause a delay in the image display. In addition, the resulting redundant memory accesses lead to an increase in the power consumption.

### SUMMARY OF THE INVENTION

The present invention has been made in view of the above inconveniences. According to an embodiment of the present invention, there are provided an image data transfer apparatus and a control method for the same that reduce redundant memory accesses leading to an increased transfer time and an increased power consumption in transferring image data as reduced images.

According to one aspect of the present invention, there is provided an image data transfer apparatus for an X-ray imaging apparatus, transferring X-ray image data output from an X-ray detection unit to an external device, comprising: an acquisition unit configured to acquire pixel values that constitute the X-ray image data in a pixel order in which the X-ray detection unit outputs the X-ray image data; a holding unit configured to divide the X-ray image data into a predetermined number of reduced images by grouping each pixel value acquired by the acquisition unit according to a pixel position in an image and to hold each reduced image in a memory area with consecutive addresses; and a transfer unit configured to transfer the X-ray image data to the external device as each reduced image held by the holding unit.

Further features of the present invention will become apparent from the following description of exemplary embodiments with reference to the attached drawings.

### BRIEF DESCRIPTION OF THE DRAWINGS

FIG. 1 is a block diagram showing an example of an X-ray imaging system in a first embodiment;

FIGS. 2A and 2B are diagrams showing a relationship between image data read from an X-ray detection unit and reduced images;

FIG. 3 is a block diagram showing an image storage control unit in FIG. 1 in detail;

FIGS. 4A and 4B are diagrams for describing differences in image data arrangement in memory between a conventional example and the present invention;

FIG. 5 is a diagram chronologically showing operations and processing in each unit according to the embodiment;

FIG. 6 is a diagram showing an exemplary order in which image data is read according to a second embodiment; and

FIG. 7 is a block diagram showing a configuration of the image storage control unit according to the second embodiment.

### DESCRIPTION OF THE EMBODIMENTS

Embodiments for Implementing the Present invention will be described in detail below with reference to the drawings.

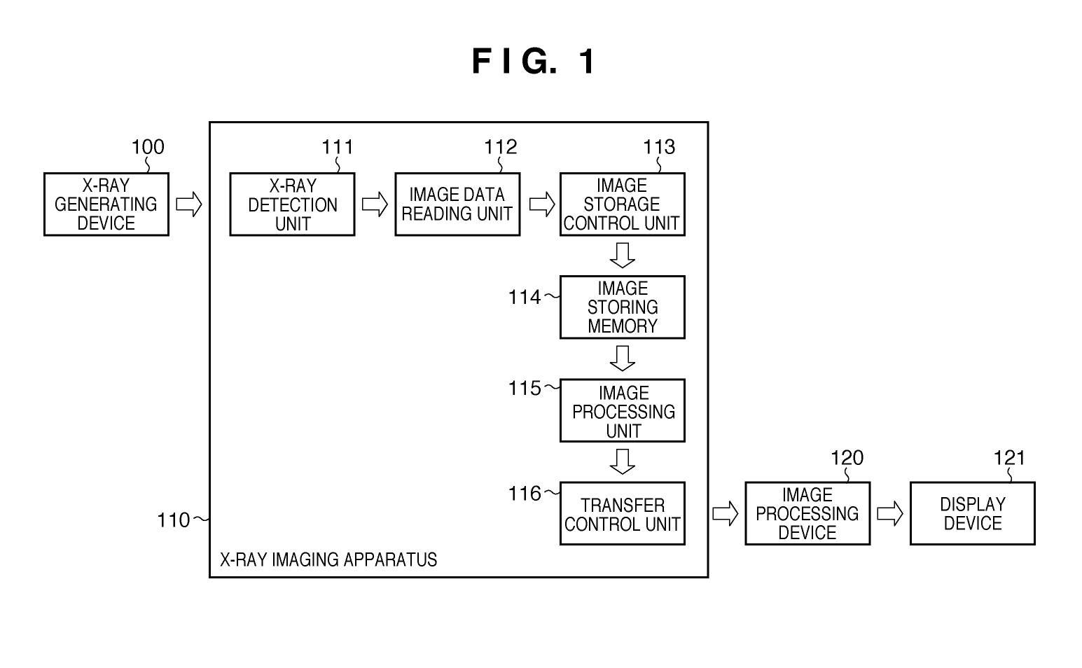

FIG. 1 is a block diagram showing an example of a general configuration in a first embodiment. As shown in FIG. 1, an X-ray imaging apparatus 110 includes an X-ray detection unit 111, an image data reading unit 112, an image storage control unit 113, an image storing memory 114, an image processing unit 115, and a transfer control unit 116.

The X-ray detection unit 111 includes a scintillator, an image pickup device array, and an A/D converter (not shown). When an X-ray is emitted from an X-ray generating device 100 toward the X-ray detection unit 111, electric signals corresponding to the amounts of X-ray incident on a fluorescent material of the scintillator are output from the image pickup devices. The electric signals are converted into digital values by the A/D converter to generate image data. The image data reading unit 112 includes a circuit (not shown) for driving the X-ray detection unit 111 and a circuit (not shown) for obtaining the digital X-ray image data output from the X-ray detection unit 111. The image data reading unit 112 obtains the X-ray image data by driving the X-ray detection unit 111 while the X-ray is emitted, and obtains offset image data by driving the X-ray detection unit 111 while the X-ray is not emitted.

The image storage control unit 113 divides the X-ray image data into a predetermined number of reduced images by grouping the obtained pixel values according to the positions of pixels in the image. That is, the image storage control unit 113 divides each of the X-ray image data and the offset image data obtained by the image data reading unit 112 into a predetermined (a plurality of) reduced images and stores each reduced image in a memory area in the image storing memory 114 allocated to the reduced image. The memory area allocated to each reduced image is an area with consecutive addresses in the image storing memory 114.

The image processing unit 115 reads the reduced X-ray image data and its corresponding reduced offset image data from the image storing memory 114 in a predetermined reduced image transfer order. The image processing unit 115 performs offset correction processing and sequentially generates image data to be transferred. The transfer control unit 116 transfers the offset-corrected image data to be transferred generated by the image processing unit 115 to an image processing device 120, which is an external device.

The image processing device 120, which is a controller that controls the X-ray imaging apparatus 110, performs image processing for the image data transferred from the X-ray imaging apparatus 110 and stores the image data. From the reduced images transferred from the X-ray imaging apparatus 110, the image processing device 120 generates a first preview, resynthesizes full image data, and as necessary, generates intermediate previews between the first preview and the full image data and performs required image processing. The generated previews and full image are displayed on a display device 121. While generating the previews and resynthesizing the original image data may be based on a known pixel synthesis scheme, the first preview is generated by using the first reduced image transferred from the X-ray imaging apparatus 110. The second preview is synthesized by using the first and second reduced images. Similarly, the full image data is resynthesized by using all the reduced images.

A wired LAN or a wireless LAN may be used for the image data transfer and other communications from the X-ray imaging apparatus 110 to the image processing device 120 performed by the transfer control unit 116. Although FIG. 1 shows the units and the data processing flow in series, this is only an illustration of a general configuration capable of implementing the functions of the present invention, and any configurations capable of implementing the functions of the present invention may be employed. For example, each unit may be interconnected as a master or slave by a known data bus.

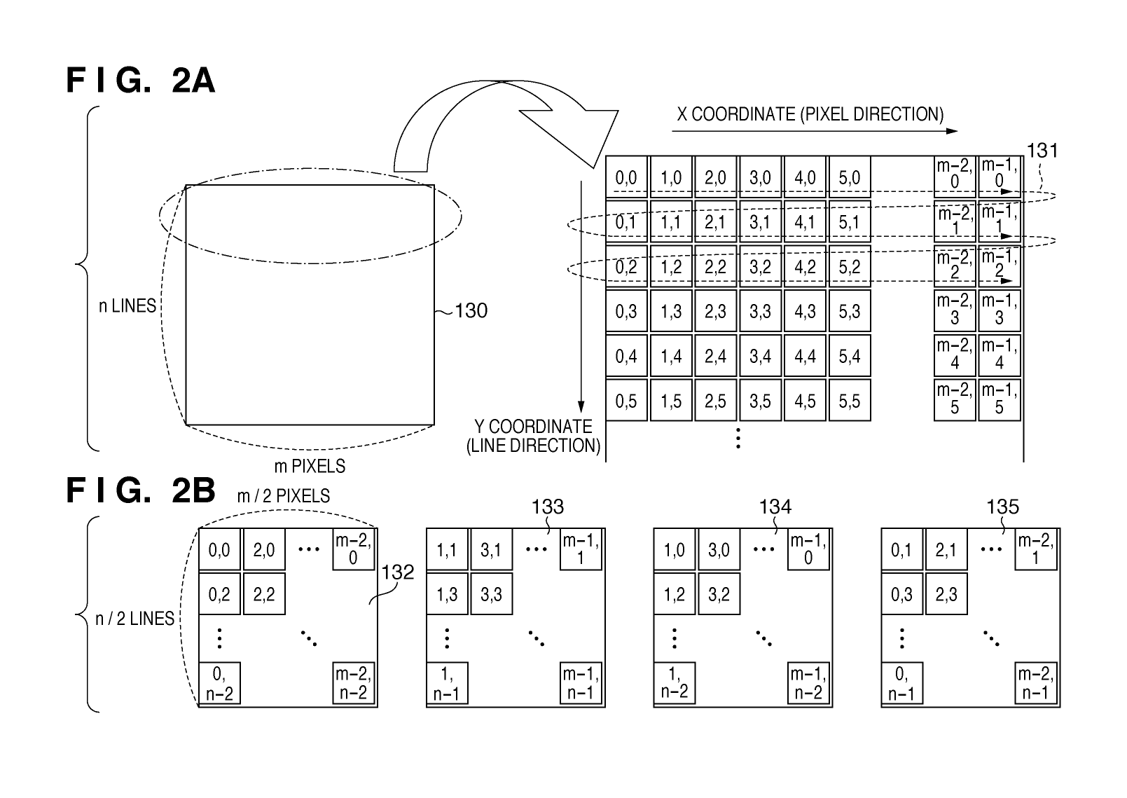

FIGS. 2A and 2B show a relationship between the original image data (also referred to as the full image data) obtained by the image data reading unit 112 and the reduced image data. FIG. 2A represents a full image 130. In this embodiment, it is assumed that the full image 130 has a size of m pixels×n lines (m and n are arbitrary natural numbers). Each block in FIG. 2A represents a pixel that constitute the full image 130, and numbers in each pixel indicate the position of the pixel in the full image as (x coordinate, y coordinate). As indicated by a pixel data reading order 131, the image data reading unit 112 reads the pixel data from the X-ray detection unit 111 in a pixel order such that the reading of the pixels one by one for one line in the x direction from the origin is repeated for all the lines in the y direction.

FIG. 2B represents reduced images 132 to 135 derived from the full image 130. In this embodiment, by way of example, the original full image is divided into four reduced images. However, the original full image may be divided into any number of reduced images, and a required configuration to be described below may be provided as appropriately according to the number of reduced images. The manner of dividing into the reduced images is generalized as follows. If image data of pixels in m columns×n rows is divided into reduced images each having a width of 1/M and a height of 1/N, where p and q are integers not smaller than 0 and k is an integer between 0 and M×N−1,

a p×M+(the remainder of k/M)-th pixel value in a q×N+(the integer part of k/M)-th line in the input image data is taken, and

the value is held as a p-th pixel value in a q-th line in a k-th reduced image.

An exemplary dividing manner for dividing the full image 130 into four to obtain the four reduced images 132 to 135 will be described below. In this case, the full image 130 is partitioned into areas of 2×2 pixels. One pixel in each area is extracted as a representative pixel of the area, so that a set of the representative pixels forms a reduced image. Since each area has four pixels, four reduced images can be generated by changing the position from which a pixel is extracted. FIG. 2B shows an example of this, in which a set of pixels with both the x and y coordinates being even numbers forms the first reduced image 132. Similarly, a set of pixels with both the x and y coordinates being odd numbers forms the second reduced image 133. A set of pixels with an odd x coordinate and an even y coordinate forms the third reduced image 134. A set of pixels with an even x coordinate and an odd y coordinate forms the fourth reduced image 135. Each of these reduced images is a reduced image thinned by ½ in height and width and has a data size of ¼ with respect to the original image data. Since the pixels that constitute the full image 130 all correspond to any of the pixels in the four reduced images 132 to 135, transferring the four reduced images 132 to 135 is equivalent to sending the full image 130 in terms of the amount of transferred data.

The above first to fourth reduced images 132 to 135 are transferred by the transfer control unit 116 to the image processing device 120 in this serial order. That is, a reduced image is sent first, followed by a reduced image having a diagonal positional relationship to the reduced image sent first. This is because a finer image can be generated by synthesizing diagonal pixels than by synthesizing vertically or horizontally adjacent pixels when two or more reduced images are synthesized to provide the second and subsequent previews. However, the above transfer order is not limiting but the reduced images may be transferred in any order.

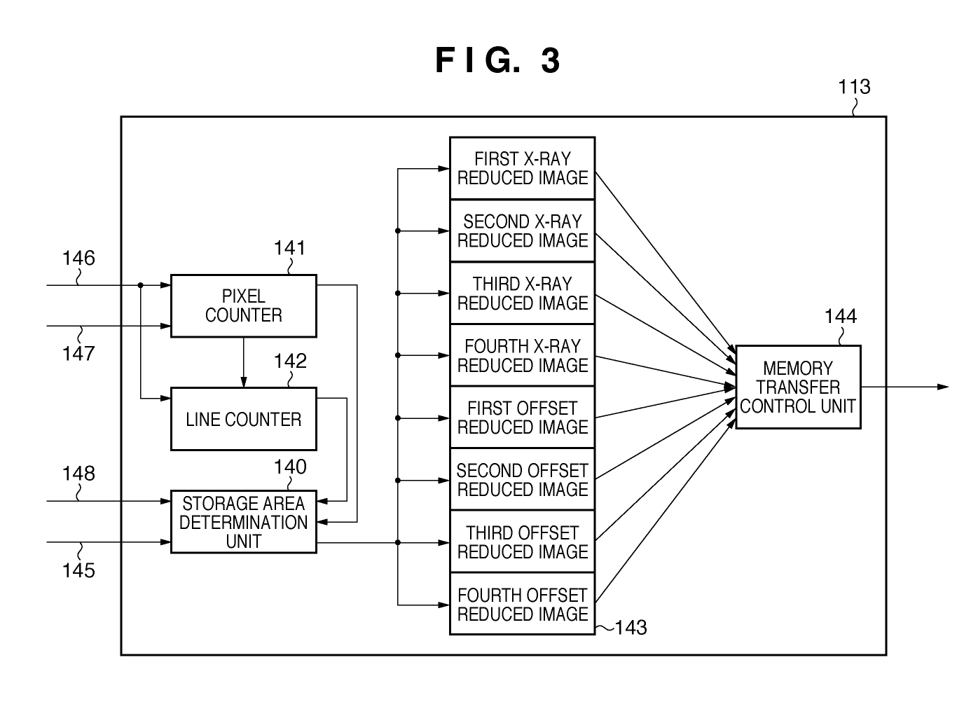

FIG. 3 shows a detailed block diagram of the image storage control unit 113 in FIG. 1. The image storage control unit 113 includes a storage area determination unit 140, a pixel (x-direction) counter 141, a line (y-direction) counter 142, a buffer 143 allocated to each reduced image, and a memory transfer control unit 144.

The following are input from the image data reading unit 112 to the image storage control unit 113:

pixel data 145 read in the pixel data reading order 131 in FIG. 2A,

a reading start signal 146 indicating the start of reading the image,

a pixel data validity signal 147 indicating whether the current pixel data 145 is valid data, and

an image data mode signal 148 indicating whether the current pixel data 145 is the X-ray image data or the offset image data.

The pixel counter 141 starts its count operation triggered by the reading start signal 146 and increments the count by one for each pixel data validity signal 147 (which is set to valid for each pixel). When the counter value reaches the number of pixels per line, the counter value is reset and the counting is repeated as above. The line counter 142 starts its count operation triggered by the reading start signal 146, and increments the count by one when the pixel counter value reaches the number of pixels per line. As illustrated in FIG. 2A, there are m pixels from 0 to (m−1) per line, and n lines from 0 to (n−1).

From the pixel counter value, the line counter value, and the image data mode signal 148, the storage area determination unit 140 determines to which reduced image the pixel data sequentially input from the image data reading unit 112 corresponds. The storage area determination unit 140 then sequentially writes the pixel data determined as above to an area in the buffer 143 allocated to the reduced image. While the configuration of the buffer 143 is not limited, the buffer 143 with a FIFO (First In First Out) configuration facilitates handling the pixel data because the pixel data will be read from the buffer 143 in the same order.

Details of the determination made by the storage area determination unit 140 are as follows. If the image data mode signal 148 indicates the X-ray image data, and

if the least significant bits of the line counter value and of the pixel counter value are both 0, the pixel data corresponds to the first X-ray reduced image,

if the least significant bits of the line counter value and of the pixel counter value are both 1, the pixel data corresponds to the second X-ray reduced image,

if the least significant bit of the line counter value is 0 and the least significant bit of the pixel counter value is 1, the pixel data corresponds to the third X-ray reduced image, and

if the least significant bit of the line counter value is 1 and the least significant bit of the pixel counter value is 0, the pixel data corresponds to the fourth X-ray reduced image.

If the image data mode signal 148 indicates the offset image data, and

if the least significant bits of the line counter value and of the pixel counter value are both 0, the pixel data corresponds to the first offset reduced image,

if the least significant bits of the line counter value and of the pixel counter value are both 1, the pixel data corresponds to the second offset reduced image,

if the least significant bit of the line counter value is 0 and the least significant bit of the pixel counter value is 1, the pixel data corresponds to the third offset reduced image, and

if the least significant bit of the line counter value is 1 and the least significant bit of the pixel counter value is 0, the pixel data corresponds to the fourth offset reduced image.

Each portion in the buffer 143 corresponding to a reduced image has associated therewith addresses of an area in the image storing memory 114 in which the corresponding reduced image is to be stored. The memory transfer control unit 144 sequentially reads the pixel data from each portion in the buffer 143 and sequentially writes the pixel data to the associated addresses in the image storing memory 114. With this configuration, each reduced image is written to and held in a memory area provided for the reduced image in the image storing memory 114 with consecutive addresses. The memory transfer control unit 144 reads the image data from each portion in the buffer 143 on the basis of a data unit efficient for the writing operation control, so that the image data is read when the amount of data for that data unit or more is accumulated in each portion in the buffer 143. The memory transfer control unit 144 then collectively writes the read image data to the image storing memory 114. Thus, efficient writing processing is achieved.

In this embodiment, the buffer 143 dedicates its areas separately to the X-ray image data and the offset image data. However, the same areas may be shared by the X-ray image data and the offset image data. In that case, for example, each portion in the buffer 143 has associated therewith both addresses of a memory area for the X-ray image data and addresses of a memory area for the offset image data. The memory transfer control unit 144 may determine, according to the image data mode signal 148, whether to write the image data to an address in the memory area for the X-ray image data or to an address in the memory area for the offset image data.

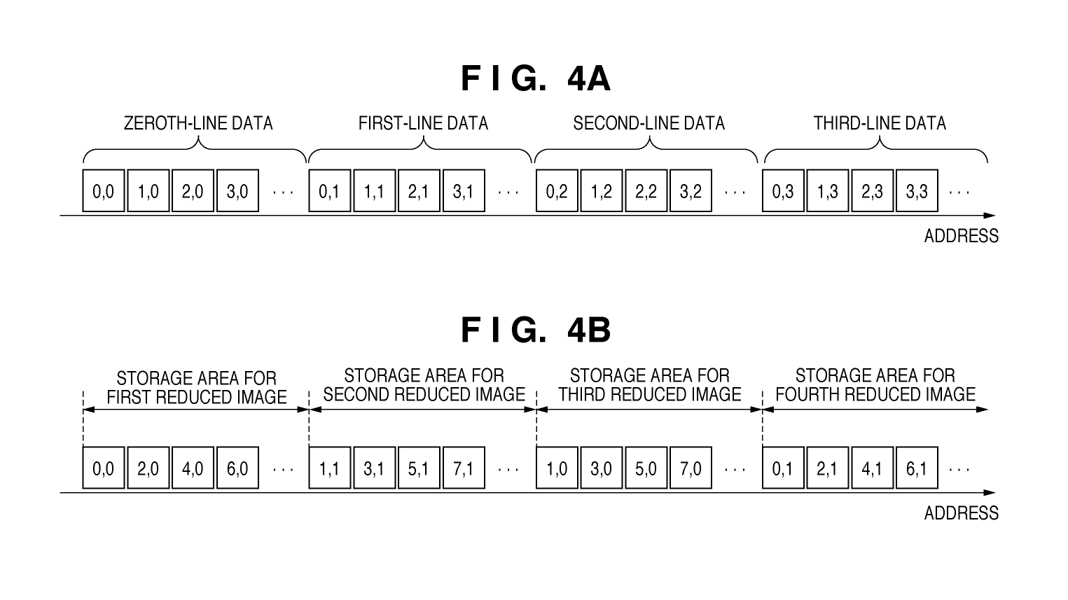

FIGS. 4A and 4B show differences in image data arrangement in memory between a conventional example and the present invention. As in FIG. 2A, each block in FIGS. 4A and 4B represents data of each pixel of a full image, and numbers in each block indicate the (x coordinate, y coordinate) in the full image.

FIG. 4A shows a pixel data arrangement in memory in the conventional example, in which the pixel data is arranged in the memory directly in the reading order 131 shown in FIG. 2A. In this case, for example, the first reduced image will be generated in the following manner:

only pixels that belong to the reduced image 1 in the even-numbered lines are read from discrete addresses, or

burst reading is performed for all the pixels in the even-numbered lines and unnecessary pixels that belong to the third reduced image are discarded.

FIG. 4B shows a data arrangement in the image storing memory 114 according to this embodiment, in which each reduced image is placed as one unit in an area for that reduced image with consecutive addresses. Thus, in this embodiment, when the image data read by the image data reading unit 112 is stored in the image storing memory 114, the image storage control unit 113 stores the image data as each reduced image rather than directly in the image data reading order 131. Accordingly, when the image data is transferred, the image processing unit 115 can simply read each reduced image in the order of the addresses.

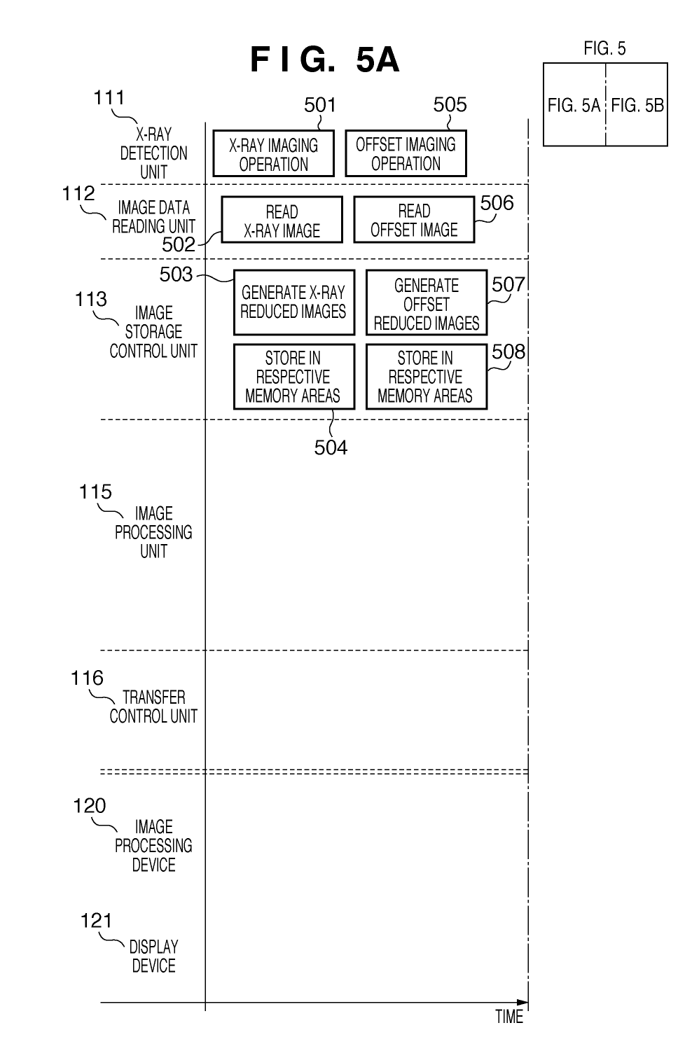

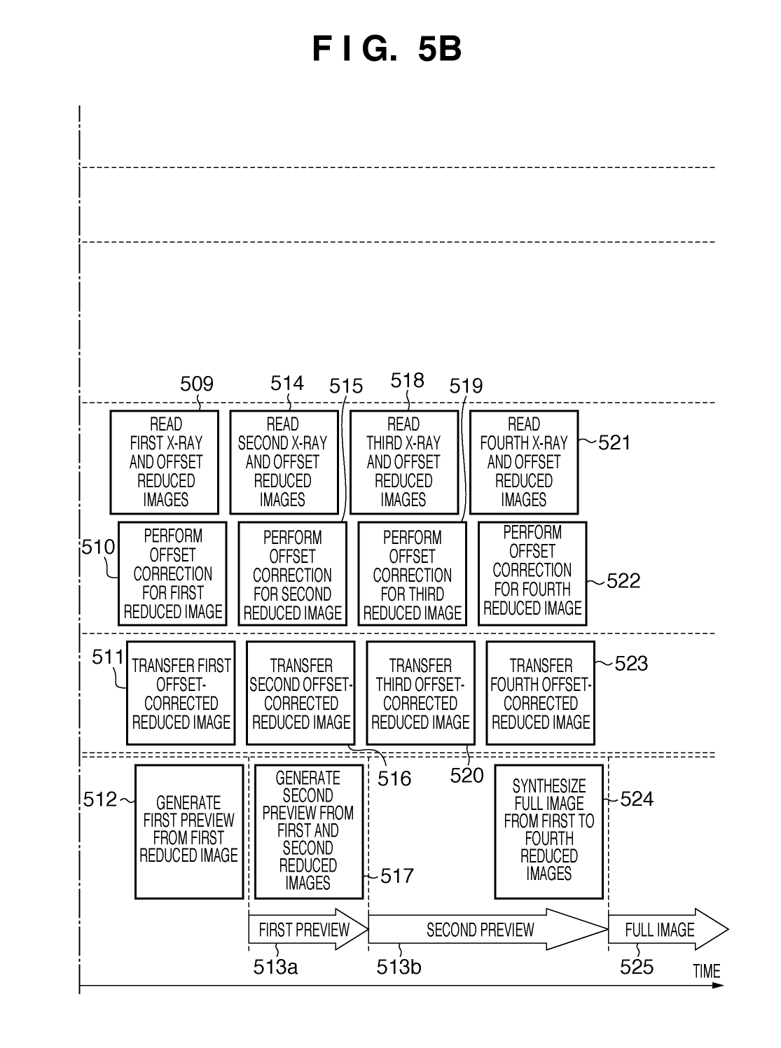

FIG. 5 chronologically shows operations and processing in each unit in this embodiment. Here, the process will be described step by step, by taking an example in which full image data is divided into four pieces of reduced image data and transferred to display a first preview, a second preview, and a full image on the display device 121.

First, an X-ray is emitted from the X-ray generating device 100 toward an object, and signals transmitted through the object are detected by the X-ray detection unit 111 (501). The image data reading unit 112 drives a reading circuit to read X-ray image data from the X-ray detection unit 111 (502). In parallel with reading the X-ray image data, the image storage control unit 113 determines to which reduced image the incoming pixel-by-pixel data belongs, with reference to the signals input from the image data reading unit 112 (503). According to the determination, the image storage control unit 113 stores the pixel data in the relevant area in the image storing memory 114. In this manner, each reduced image is generated in the image storing memory 114 (504).

Following the obtainment of the X-ray image data, image data with no X-ray irradiation, i.e., offset image data, is obtained through processing similar to the above (505 to 508). In this embodiment, data transfer to the image processing device 120 during the reading of the image data may cause noises in the read image data due to the data transfer. For this reason, processing in the image processing unit 115 and the subsequent units (processing in 509 and the subsequent steps) is not performed during the reading of the image data (during the processing in 501 to 508).

Once the image data has been read and the reduced images of the X-ray image and of the offset image have been generated in the image storing memory 114, the image processing unit 115 reads the first X-ray reduced image and the first offset reduced image from the image storing memory 114 (509). The image processing unit 115 uses the first offset reduced image to perform offset correction processing for the first X-ray reduced image (510), thereby obtaining a first corrected reduced image. The offset correction processing is performed by subtracting the value of each pixel in the offset reduced image from the value of each corresponding pixel in the X-ray reduced image.

The transfer control unit 116 transfers the first corrected reduced image to the image processing device 120 (511). The image processing device 120 performs required image processing for the received first corrected reduced image to generate a first preview (512), which is sent to the display device 121 to be displayed thereon (513a).

Through similar processing, a second corrected reduced image is transferred to the image processing device 120 (514 to 516). The image processing device 120 uses the second corrected reduced image and the previously received first corrected reduced image to perform synthesis and required image processing, thereby generating a finer second preview with an amount of information larger than that of the first preview (517). The image processing device 120 sends the second preview to the display device 121 to be displayed thereon (513b).

Through similar processing, a third reduced image and a fourth reduced image are sequentially transferred to the image processing device 120 (518 to 523). The image processing device 120 uses all the received first to fourth reduced images to perform synthesis and required image processing, thereby generating and storing a full image (524). The image processing device 120 sends the full image to the display device 121 to be displayed thereon (525).

According to the above image data transferring process in the first embodiment, the image processing unit 115 can efficiently read the reduced images, resulting in reductions in the reading time and in redundant memory accesses. This can contribute to reductions in the time for the images to be displayed and in the power consumption.

Next, a second embodiment of the present invention will be described.

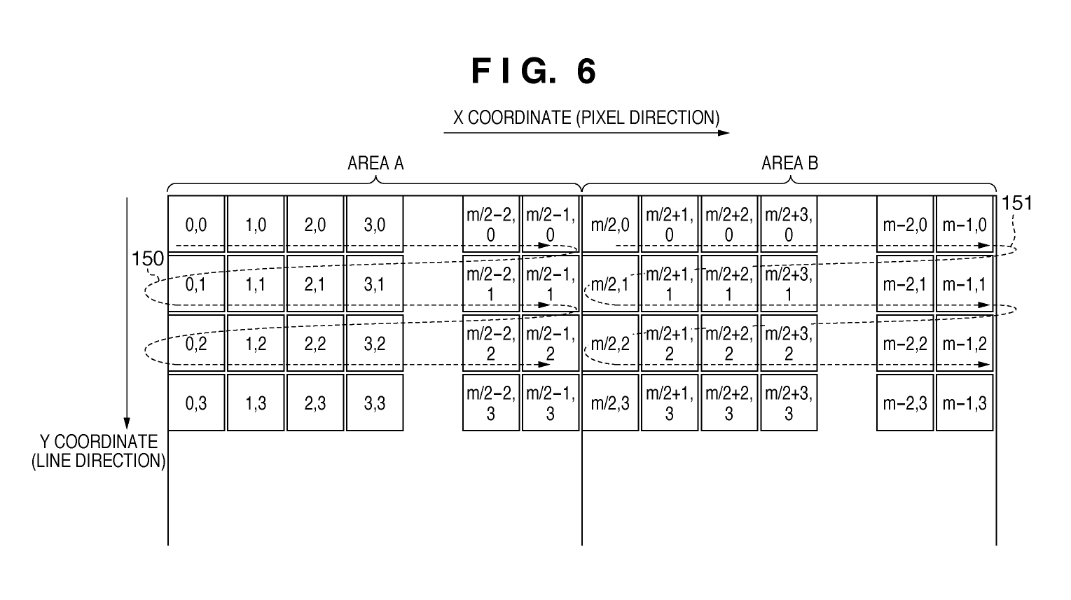

The first embodiment has illustrated the case in which the image data reading unit 112 reads the pixel data from the X-ray detection unit 111 in the reading order 131 shown in FIG. 2A. In the second embodiment to be described, the image data reading unit 112 divides the full image into two partial areas A and B in the pixel direction as shown in FIG. 6, for example, and reads the pixel values from the two partial areas in parallel. Since the two areas in the full image can be read in parallel in reading orders 150 and 151, the reading time can be reduced.

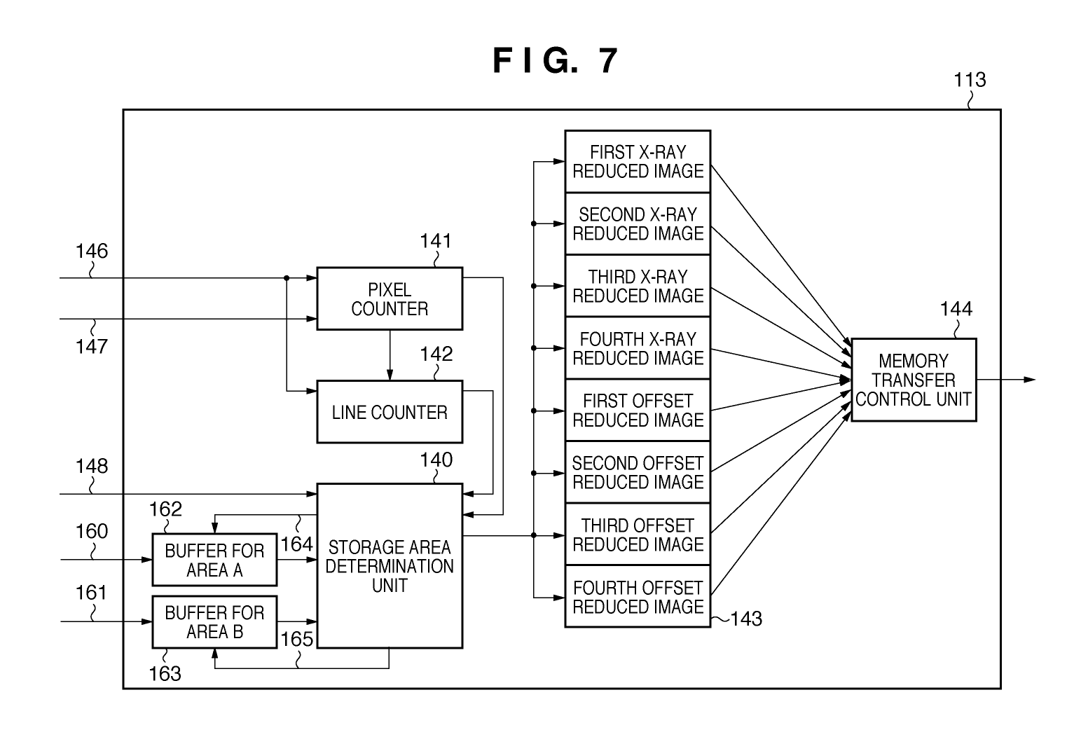

The parallel reading as in FIG. 6 can be supported by configuring the image storage control unit 113 as in FIG. 7. Pixel data 160 in the area A and pixel data 161 in the area B are read from these two areas in the reading orders 150 and 151 as in FIG. 6, and written to a buffer 162 for the area A and a buffer 163 for the area B in the reading orders, respectively. While the pixel counter value indicates a pixel in the area A, that is, while the counter value is between 0 and m/2−1, the storage area determination unit 140 outputs a buffer reading signal 164 for the area A to sequentially obtain the pixel data in the area A from the buffer 162 for the area A. Thereafter, through processing as in the first embodiment, the pixel data is held in the buffer 143 as any of the first to fourth X-ray reduced images or any of the first to fourth offset reduced images.

When the pixel counter value indicates a pixel in the area B, that is, when the counter value shows a value between m/2and m −1, the storage area determination unit 140 stops outputting the buffer reading signal 164 for the area A and outputs a buffer reading signal 165 for the area B. This causes the pixel data in the area B to be sequentially obtained from the buffer 163 for the area B. Thereafter, through processing as in the first embodiment, the pixel data is held in the buffer 143 as any of the first to fourth X-ray reduced images or any of the first to fourth offset reduced images. When the pixel counter value again indicates a pixel in the area A, or when the line counter value is incremented, it means that the reading proceeds to the next line. Then the process of reading from the buffer 162 for the area A is performed.

According to the above process, the present invention can also be applied to the parallel reading from two areas. The manner of dividing the image is not limited to the example in FIG. 6, but the image may be divided into any number of areas and in any ratio. Further, besides in the pixel direction, the image may be divided in the line direction.

Thus, according to the above embodiments, the X-ray image data is stored in the form of pieces of reduced image data in respective areas in memory (areas with consecutive addresses). This eliminates redundant memory accesses, enabling the reduced image data to be efficiently read when transferred. Thus, an optimal method can be provided for realizing an X-ray imaging apparatus, which requires displaying a preview image in a short time after reduced images are transferred.

In the above embodiments, the image data is reduced by ½ in height and width and held as the four reduced images. However, the reduction factor in height and width is not limited to this. Further, different reduction factors may be set for the height and the width, respectively, so that the image may be reduced by 1/M in width and by 1/N in height to obtain M×N reduced images (M and N are integers greater than 1). In this case, the pixel value of an m-th pixel (m, n) in an n-th line in a k-th reduced image held in the image storing memory 114 is the pixel value of an m×M+(the remainder of k/M)-th pixel in an n×N+(the integer part of e,fra k/M)-th line in the input image data, where n and m are integers not smaller than 0, and k is an integer between 0 and M×N−1.

While the above embodiments have been described for the cases of applying the image data transfer apparatus of the present invention to an X-ray imaging apparatus, this is not limiting. Rather, application to transferring an image obtained by a general imaging sensor is of course possible. If noises due to image data transfer do not affect image data read from the imaging sensor, the transfer control unit 116 may transfer the image while the image data reading unit 112 is reading the image data.

According to the present invention, reduced images of image data are held in areas in memory with consecutive addresses. This can reduce redundant memory accesses that lead to an increased transfer time and an increased power consumption in data transfer of the reduced images.

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (e.g., non-transitory computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-179010, filed Aug. 9, 2010, which is hereby incorporated by reference herein in its entirety.

What is claimed is:

1. An image data transfer apparatus for an X-ray imaging apparatus, transferring X-ray image data output from an X-ray detection unit to an external device, comprising:
an acquisition unit configured to acquire pixel values that constitute the X-ray image data in a pixel order in which the X-ray detection unit outputs the X-ray image data;
a holding unit configured to divide the X-ray image data into a predetermined number of reduced images by grouping each pixel value acquired by said acquisition unit according to a pixel position in an image and to hold each reduced image in a memory area with consecutive addresses; and
a transfer unit configured to transfer the X-ray image data to the external device as each reduced image held by said holding unit.

2. The apparatus according to claim 1, wherein
said holding unit holds, as the predetermined number of X-ray reduced images, the X-ray image data acquired by said acquisition unit with X-ray irradiation, and holds, as the predetermined number of offset reduced images, offset image data acquired by said acquisition unit without X-ray irradiation, and
said transfer unit generates corrected X-ray reduced images by correcting each X-ray reduced image with each corresponding offset reduced image and transfers the image data to the external device as each corrected X-ray reduced image.

3. The apparatus according to claim 2, wherein
said transfer unit transfers the image data as each corrected reduced image after the X-ray image data of an image and the offset image data of the image acquired by said acquisition unit are held by said holding unit as the predetermined number of X-ray reduced images and the predetermined number of offset reduced images.

4. The apparatus according to claim 1, wherein
the X-ray detection unit divides an image into partial areas and outputs the pixel values in parallel from each partial area,
the acquisition unit acquires the pixel values output in parallel from the partial areas and holds the pixel values in a buffer, and
the holding unit divides the image data held in the buffer into the reduced images and holds the reduced images.

5. The apparatus according to claim 1, wherein
said transfer unit sends a reduced image first, and then sends a reduced image having a diagonal positional relationship to the reduced image sent first.

6. An image data transfer apparatus transferring image data output from an imaging sensor to an external device, comprising:
an acquisition unit configured to acquire pixel values that constitute the image data in a pixel order in which the imaging sensor outputs the image data;
a holding unit configured to divide the image data into a predetermined number of reduced images by grouping each pixel value acquired by said acquisition unit according to a pixel position in an image and to hold each reduced image in a memory area with consecutive addresses; and
a transfer unit configured to transfer the image data to the external device as each reduced image held by said holding unit.

7. The apparatus according to claim 6, wherein
the pixel order is an order such that pixels are horizontally input one by one and lines are vertically input one by one in the image represented by the image data,
the predetermined number of reduced images are M ×N reduced images resulting from reducing the image data by 1/M in width and by 1/N in height (M and N are integers greater than 1), and
a p×M+(a remainder of k/M)-th pixel value in a q×N+(an integer part of k/M)-th line in the image data acquired by said acquisition unit is held by said holding unit as a p-th pixel value in a q-th line in a k-th reduced image (p and q are integers not smaller than 0, and k is an integer between 0 and M×N−1).

8. A control method for an image data transfer apparatus for an X-ray imaging apparatus, transferring X-ray image data output from an X-ray detection unit to an external device, comprising:
an acquisition step of acquiring pixel values that constitute the X-ray image data in a pixel order in which the X-ray detection unit outputs the X-ray image data;
a holding step of dividing the X-ray image data into a predetermined number of reduced images by grouping each pixel value acquired in said acquisition step according to a pixel position in an image and of holding each reduced image in a memory area with consecutive addresses; and
a transfer step of transferring the X-ray image data to the external device as each reduced image held in the memory area in said holding step.

9. A control method for an image data transfer apparatus transferring image data output from an imaging sensor to an external device, comprising:
an acquisition step of acquiring pixel values that constitute the image data in a pixel order in which the imaging sensor outputs the image data;
a holding step of dividing the image data into a predetermined number of reduced images by grouping each pixel value acquired in said acquisition step according to a pixel position in an image and of holding each reduced image in a memory area with consecutive addresses; and
a transfer step of transferring the image data to the external device as each reduced image held in the memory area in said holding step.

10. A non-transitory computer readable medium having stored therein a program for causing a computer to perform each step of the control method for an image data transfer apparatus according to claim 8.

11. A non-transitory computer readable medium having stored therein a program for causing a computer to perform each step of the control method for an image data transfer apparatus according to claim 9.
